(19) United States
(12) Patent Application Publication
(10) Pub. No.: US 2024/0216063 A1
(43) Pub. Date: Jul. 4, 2024

(54) System and Method for Providing Guidance Itinerary for Interventional Medical Procedures

(71) Applicant: GE Precision Healthcare LLC, Wauwatosa, WI (US)

(72) Inventors: Yves Trousset, Palaiseau (FR); Rémi Gerfault, Cholet (FR)

(21) Appl. No.: 18/089,905

(22) Filed: Dec. 28, 2022

(51) Int. Cl. A61B 34/10

(52) U.S. Cl. CPC A61B 34/10; A61B 2034/105; A61B 2034/107

(57) ABSTRACT

A 3D/2D imaging system and method analyzes various anatomical structures, such as the organs and/or vascular structures within the imaged anatomy through which the interventional device can pass to reach the target tissue. The imaging system can determine the locations, characteristics, and configurations of the vascular structures/blood vessels and/or organs. During the performance of the interventional procedure, the information provided by the imaging system from the 3D volume can be employed to optimally position the intra-operative imaging device in order to obtain a desired visualization e.g., 2D view, of the position of the interventional device within the patient anatomy. This intra-operative 2D view is optionally registered to the 3D volume and can be displayed by the imaging system along with a 3D model or image determined from the 3D volume that is representative of the patient anatomy present in the intra-operative 2D image.

### FIELD OF THE DISCLOSURE

The invention relates generally to navigation of medical instruments in a medical procedure, and in particular, systems and methods to locate and direct the movement of medical instruments within a patient anatomy during the medical procedure.

### BACKGROUND OF THE DISCLOSURE

Image-guided surgery is a developing technology that allows surgeons to perform an intervention or a surgery in a minimally invasive way while being guided by images, which may be “real” images or virtual images. For instance, in laparoscopic surgery, a small video camera is inserted through a small incision made in the patient skin. This video camera provides the operator with a “real” image of the anatomy. In other types of image-guided surgery, such as endo-vascular surgery where a lesion is treated with devices inserted through a catheter navigated into the arteries of the patient, are “image-guided” because low dose x-ray images (also called fluoroscopy images) and/or ultrasound (US) images are used to guide the catheters and the devices through the patient anatomy. The fluoroscopy/US image is a “real” image, not a virtual image, as it is obtained using real X-rays or ultrasound waves and shows the real anatomy of the patient. Then there are also cases where a “virtual” image” is used, which is a combination of real images utilized to form the virtual image of the anatomy in a known manner. An example of image-guided surgery using both “real” and “virtual” images is the minimally invasive surgery of the heart or spine, where “real” fluoroscopy and/or US images acquired during the surgery are used to guide the insertion of devices in the vascular structures or vertebrae, while pre-operative CT or Cone-beam CT (CBCT) images are also used, in conjunction with surgical navigation systems, to visualize the location of the devices in the 3D anatomy of the patient. Because the display of the location of the devices in the CT or CBCT images is not the result of a direct image acquisition performed during the surgery, but from a combination of pre-existing real images and information provided by the surgical navigation system, the display of the device location in the CT or CBCT images is described as a “virtual” image.

Regardless of particular images utilized in its formation, image-guided surgery allows the surgeon to reduce the size of entry or incision into the patient, which can minimize pain and trauma to the patient and result in shorter hospital stays. Examples of image-guided procedures include laparoscopic surgery, thorasoscopic surgery, endoscopic surgery, etc. Types of medical imaging systems, for example, radiologic imaging systems, computerized tomography (CT), magnetic resonance imaging (MRI), positron emission tomography (PET), ultrasound (US), X-ray angiography machines, etc., can be useful in providing static image guiding assistance to medical procedures. The above-described imaging systems can provide two-dimensional or three-dimensional images that can be displayed to provide a surgeon or clinician with an illustrative map to guide a tool (e.g., a catheter) through an area of interest of a patient's body.

In clinical practice, minimally invasive percutaneous cardiac and vascular interventions are becoming more prevalent as compared with traditional open surgical procedures. Such minimally invasive percutaneous cardiac and vascular interventions have advantages of shorter patient recovery times, as well as faster and less risky procedures. In such minimally invasive cardiac and vascular interventions, devices such as stents or stent grafts are delivered into the patient through vessels via a catheter. Navigating the catheter inside the vessels of a patient is challenging.

More recently, solutions for easing the navigation of the catheter have been developed that are based on the fusion of a pre-operative 3D computed tomography (CT) image that shows the anatomy of the patient through which the interventional tool is to be navigated with the fluoroscopy and/or US images to improve the guidance for an interventional procedure. Ultrasound images include more anatomical information of cardiac structures than x-ray images which do not effectively depict soft structures, while x-ray images more effectively depict catheters and other surgical instruments than ultrasound images. In this process, as shown in FIG. 1, initially a pre-op CT image 1000 is obtained, which normally takes the form of a 3D volume of the anatomy of the patient. Subsequently, an intra-operative image 1002 is obtained of the patient during the procedure, and the intra-operative image 1002 is registered to the 3D volume in order to determine a 2D image within the 3D volume along the same image plane as the intra-operative image 1002, thus creating the pre-op image 1000. The pre-op image 1000 and the intra-operative image 1002 can each be displayed to the physician performing the procedure, such as by overlaying the intra-operative image 1002 onto the pre-op image 1000 or vice versa, to illustrate a fusion image 1004 illustrating both the structure of the anatomy of the patient and the current location of the interventional tool, e.g., a guide wire or catheter, within the anatomy. These fusion image solutions, which can employ fluoro/X-ray or ultrasound images as the intra-operative images, can more clearly illustrate the interventional tool location within the patient anatomy.

However, while this image combination provides the physician with the ability to interpret the differences in the displayed anatomies, it is completely left to the experience and discretion of the physician to utilize the displayed information to identify the displayed patient anatomy in each if the respective CT and intra-operative images. In particular, the fusion image 1004 provides only a 2D representation of the anatomy and the interventional device, e.g., catheter, which does not provide the depth dimension for the anatomy, such that certain relevant portions of the anatomy can be obscured due to other portions of the anatomy being overlaid thereon.

Further, with regard to the overall procedure being performed, in many procedures the path from the point of the incision to the target tissue within the patient extends through many different vascular structures and/or other tissues. While the image combination provides information regarding the blood vessel or structure in which the interventional device is currently positioned, this is the extend of the information provided by the images. Thus, with regard to each bifurcation of a blood vessel or other structure through which the interventional device passes along the path to the target tissue, the physician must make continual decisions regarding the proper branch in which to move the interventional device to follow the path. While it has been proposed to enable pre-operative planning and annotations for the path to be taken by the interventional device to reach the target tissue, such as that disclosed in US Patent Application Publication No. US2018/0235701, entitled Systems And Methods For Intervention Guidance Using Pre-Operative Planning With Ultrasound, the entirety of which is expressly incorporated by reference herein for all purposes, the pre-planning annotations regard the steps or itinerary for the planned procedure remain displayed in conjunction with the 2D image/image combination that lacks the depth to enable the physician to readily discern the proper path to take with regard to the blood vessel or other tissue and/or vascular structure displayed in the 2D image.

As a result, it is desirable to develop an imaging system and method that can improve upon existing systems and methods to provide an enhanced visualization of the patient, e.g., organs and/or vascular structure or blood vessels, through which a physician is navigating an interventional device during a medical procedure.

### BRIEF DESCRIPTION OF THE DISCLOSURE

The above-mentioned drawbacks and needs are addressed by the embodiments described herein in the following description.

According to one aspect of an exemplary embodiment of the invention, an imaging system is utilized to obtain pre-operative images of the anatomy of a patient in order to provide a navigational roadmap of for the insertion of an interventional tool, e.g., a guide wire or catheter, into and through the anatomy. The imaging system creates a 3D volumetric image of the anatomy and analyzes the 3D volume to assist the physician in planning the path for the insertion of the interventional device through the patient to the target tissue,(s), e.g., a tumor, embolization, and/or tissue for biopsy, on which the interventional procedure is to be performed.

By itself, or in conjunction with a manual, annotating review of the 3D volume by the physician, the imaging system can analyze the various anatomical structures, such as the organs and/or vascular structures within the imaged anatomy through which the interventional device can pass to reach the target tissue. In the analysis, the imaging system can determine the locations and configurations of the vascular structures/blood vessels and/or organs, including the location of angles and/or bifurcations of the passages within the organs and/or blood vessels, the diameter and tortuosity of the passages within the organs and/or blood vessels. With this information the imaging system can provide suggestions to the physician regarding an optimized path to the target tissue, along with the various steps to be taken along the path relative to the detected blood vessel structures. In addition, the imaging system can provide suggestions on the type of interventional device best suited for performing the procedure based on the configuration of the vascular structures/blood vessels constituting the optimized path to the target tissue.

In addition, during the performance of the interventional procedure, the information provided by the imaging system on the 3D volume can be employed to optimally position the intra-operative imaging device in order to obtain a desired visualization e.g., 2D view, of the position of the interventional device within the patient anatomy. This intra-operative 2D view is registered to the 3D volume and can be displayed by the imaging system along with a 3D model or image determined from the 3D volume that is representative of the patient anatomy present in the intra-operative 2D image. With the 3D model or image presented along with the intra-operative 2D image, the physician is presented with a reference illustrating the 3D orientation of the vascular structure presented in the intra-operative image, allowing the physician to more readily navigate the interventional device along the predetermined route. Further, for each successive intra-operative 2D view that is obtained during the interventional procedure, the 2D view is registered to the 3D volume and presented to the physician along with the 3D image determined from the 3D volume representative of the patient anatomy present in the current intra-operative 2D image.

According to still a further aspect of one exemplary embodiment of the disclosure, a method for providing guidance for an interventional device during an interventional medical procedure includes the steps of obtaining a pre-operative 3D image volume of a patient anatomy utilizing a first imaging system, identifying one or more structures, characteristics of the one or more structures, and at least one target tissue in the image volume, planning an itinerary including a number of steps for insertion of an interventional device through the patient anatomy to the target tissue, obtaining an intra-operative 2D image of the patient anatomy and interventional device according to one step of the itinerary utilizing a second imaging system and registering the intra-operative 2D image to the 3D image volume.

According to still a further aspect of one exemplary embodiment of the disclosure, an imaging system for providing guidance for movement of an interventional device in an interventional medical procedure includes a first imaging system for obtaining a pre-operative 3D image volume of a patient anatomy, a second imaging system for obtaining an intra-operative 2D image of the patient anatomy and a computing device operably connected to the first imaging system and to the second imaging system, the computing device configured to identify one or more structures, characteristics of the one or more structures, and at least one target tissue in the image volume, to plan an itinerary including a number of steps for insertion of an interventional device through the patient anatomy to the target tissue, and to register the intra-operative 2D image to the 3D image volume.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The drawings illustrate the best mode presently contemplated of carrying out the disclosure. In the drawings:

FIG. 1 is a schematic drawing of an imaging system according to one exemplary embodiment of the disclosure.

FIG. 2 is flowchart illustrating the method of operation of the imaging system in performing an interventional medical procedure according to one exemplary embodiment of the disclosure.

FIG. 3 is a diagrammatic representation of a display screen presented during the performance of an interventional medical procedure employing the imaging system according to an exemplary embodiment of the disclosure.

### DETAILED DESCRIPTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

The following description presents embodiments of systems and methods for imaging patient anatomy in real-time during interventional and/or surgical procedures. Particularly, certain embodiments describe systems and methods for imaging processes for updating images illustrating the patient anatomy during minimally-invasive interventional procedures. The interventional procedures, for example, may include angioplasty, stent placement, removal of blood clots, localized thrombolytic drug administration, perfusion studies, balloon septostomy, Transcatheter Aortic-Valve Implantation (TAVI), EVAR, tumor embolization and/or an electrophysiology study.

It may be noted that in the present description, the terms “dynamic process(s)” and “transient phenomena” have been used interchangeably to refer to processes and events where at least a portion of the subject to be imaged exhibits motion or other dynamic processes over time, such as, movement of an interventional device through a vascular structure. By way of example, the dynamic processes may include fluid flow through a passage, device vibrations, take-up and wash-out of a contrast medium, cardiac motion, respiratory motion, peristalsis, and/or change in tissue perfusion parameters including regional blood volume, regional mean transit time and/or regional blood flow.

Additionally, the following description presents embodiments of imaging systems, such as radiologic imaging systems, and methods that minimize contrast agent dosage, x-ray radiation exposure and scan durations. Certain embodiments of the present systems and methods may also be used for reconstructing high-quality 3D cross-sectional images in addition to the 2D projection images for allowing diagnosis, therapy delivery, and/or efficacy assessment.

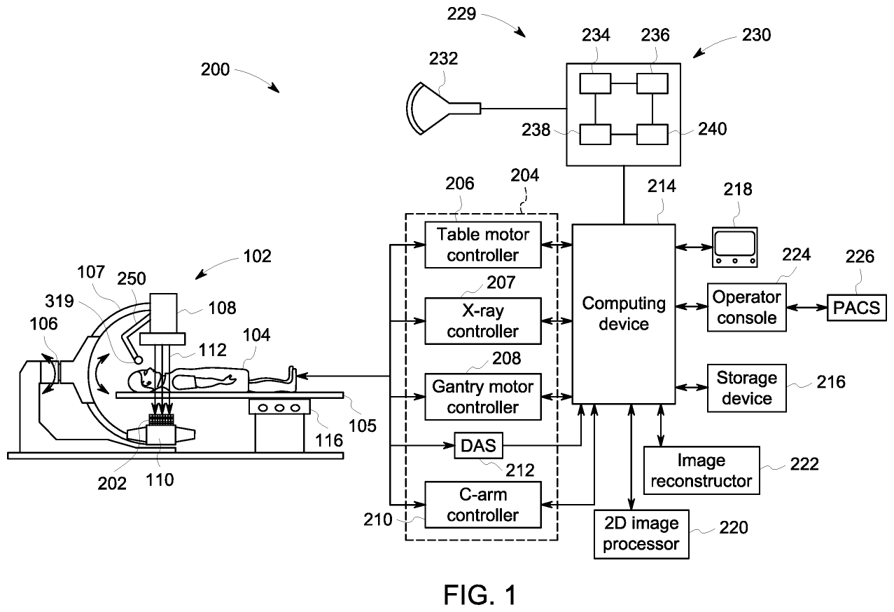

For discussion purposes, embodiments of the present systems are described with reference to use of a C-arm system employing conventional and unconventional acquisition trajectories for imaging a target region of the subject. In certain embodiments, the present systems and methods may be used during interventional or surgical procedures. Additionally, embodiments of the present systems and methods may also be implemented for imaging various transient phenomena in non-medical imaging contexts, such as security screening and/or industrial nondestructive evaluation of manufactured parts. An exemplary system that is suitable for practicing various implementations of the present technique is described in the following section with reference to FIG. 1.

FIG. 1 illustrates an exemplary radiologic imaging system 200, for example, for use in interventional medical procedures, such as that disclosed in U.S. Pat. No. 10,524,865, entitled Combination Of 3D Ultrasound And Computed Tomography For Guidance In Interventional Medical Procedures, the entirety of which is expressly incorporated herein by reference for all purposes. In one embodiment, the system 200 may include a C-arm radiography system 102 configured to acquire projection data from one or more view angles around a subject, such as a patient anatomy 104 positioned on an examination table 105 for further analysis and/or display. To that end, the C-arm radiography system 102 may include a gantry 106 having a mobile support such as a movable C-arm 107 including at least one radiation source 108 such as an x-ray tube and a detector 110 positioned at opposite ends of the C-arm 107. In exemplary embodiments, the radiography system 102 can be an x-ray system, a positron emission tomography (PET) system, a computerized tomosynthesis (CT) system, an angiographic or fluoroscopic system, and the like or combination thereof, operable to generate static images acquired by static imaging detectors (e.g., CT systems, MRI systems, etc.) prior to a medical procedure, or real-time images acquired with real-time imaging detectors (e.g., angioplastic systems, laparoscopic systems, endoscopic systems, etc.) during the medical procedure, or combinations thereof. Thus, the types of acquired images can be diagnostic or interventional.

In certain embodiments, the radiation source 108 may include multiple emission devices, such as one or more independently addressable solid-state emitters arranged in one or multi-dimensional field emitter arrays, configured to emit the x-ray beams 112 towards the detector 110. Further, the detector 110 may include a plurality of detector elements that may be similar or different in size and/or energy sensitivity for imaging a target tissue 317 or other region of interest (ROI) of the patient anatomy 104 at a desired resolution.

In certain embodiments, the C-arm 107 may be configured to move along a desired scanning path for orienting the x-ray source 108 and the detector 110 at different positions and angles around the patient anatomy 104 for acquiring information for 3D imaging of dynamic processes. Accordingly, in one embodiment, the C-arm 107 may be configured to rotate about a first axis of rotation. Additionally, the C-arm 107 may also be configured to rotate about a second axis in an angular movement with a range of about plus or minus 60 degrees relative to the reference position. In certain embodiments, the C-arm 107 may also be configured to move forwards and/or backwards along the first axis and/or the second axis.

Accordingly, in one embodiment, the C-arm system 102 may include control circuitry 114 configured to control the movement of the C-arm 107 along the different axes based on user inputs and/or protocol-based instructions. To that end, in certain embodiments, the C-arm system 102 may include circuitry such as tableside controls 116 that may be configured to provide signals to the control circuitry 114 for adaptive and/or interactive control of imaging and/or processing parameters using various input mechanisms. The imaging and/or processing parameters, for example, may include display characteristics, x-ray technique and frame rate, scanning trajectory, table motion and/or position, and gantry motion and/or position.

In certain embodiments, the detector 110 may include a plurality of detector elements 202, for example, arranged as a 2D detector array for sensing the projected x-ray beams 112 that pass through the patient anatomy 104. In one embodiment, the detector elements 206 produce an electrical signal representative of the intensity of the impinging x-ray beams 112, which in turn, can be used to estimate the attenuation of the x-ray beams 112 as they pass through the patient anatomy 104. In another embodiment, the detector elements 202 determine a count of incident photons in the x-ray beams 112 and/or determine corresponding energy.

Particularly, in one embodiment, the detector elements 202 may acquire electrical signals corresponding to the generated x-ray beams 112 at a variety of angular positions around the patient anatomy 104 for collecting a plurality of radiographic projection views for construction of X-ray images, such as to form fluoro image(s). To that end, control circuitry 114 for the system 200 may include a control mechanism 204 configured to control position, orientation and/or rotation of the table 105, the gantry 106, the C-arm 107 and/or the components mounted thereon in certain specific acquisition trajectories.

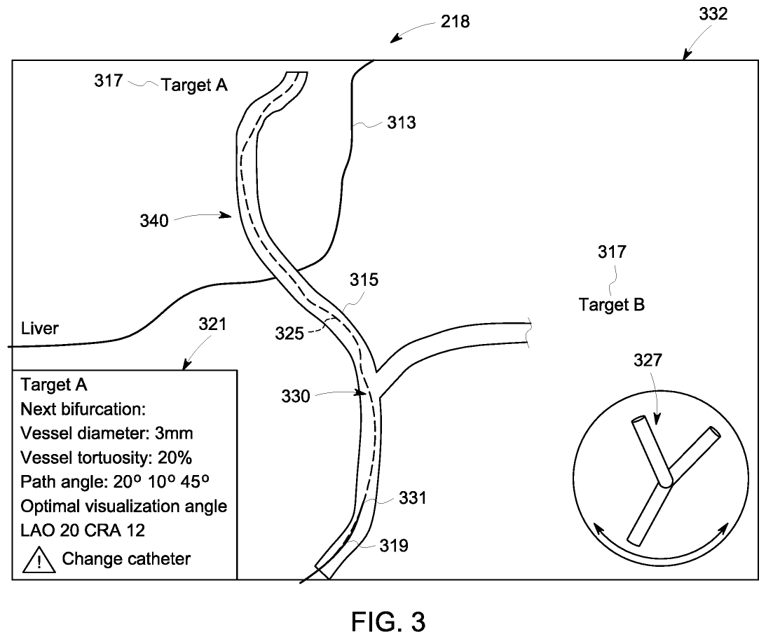

The control mechanism 204, for example, may include a table motor controller 206, which allows control of the position and/or orientation of the table 105 based on a protocol-based instruction and/or an input received from the physician, for example, via tableside controls, such as a joystick. During an intervention, for example, the physician may grossly position an interventional device 319 (FIG. 3) in the patient anatomy 104 in the field of view of the system 102 by moving the table 105 using the table motor controller 206. Once the interventional device can be visualized, the physician may advance position of the interventional device 319 within the vasculature and performs an interventional diagnostic or therapeutic procedure.

In certain embodiments, the x-ray source 108 and the detector 110 for interventional imaging may be controlled using an x-ray controller 207 in the control mechanism 204, where the x-ray controller 207 is configured to provide power and timing signals to the radiation source 108 for controlling x-ray exposure during imaging. Further, the control mechanism 204 may also include a gantry motor controller 208 that may be configured to control the rotational speed, tilt, view angle, and/or position of the gantry 106. In certain embodiments, the control mechanism 204 also includes a C-arm controller 210, which in concert with the gantry motor controller 208, may be configured to move the C-arm 107 for real-time imaging of dynamic processes.

In one embodiment, the control mechanism 204 may include a data acquisition system (DAS) 212 for sampling the projection data from the detector elements 206 and converting the analog data to digital signals for image reconstruction by 2D image processor 220, for reconstructing high-fidelity 2D images in real-time for use during the interventional procedure, and/or 3D image processor/reconstructor 222, for generating 3D cross-sectional images (or 3D volumes), and subsequent illustration of the images on display 218. Moreover, in certain embodiments, the data sampled and digitized by the DAS 212 may be input to a system controller/processing unit/computing device 214. Alternatively, in certain embodiments, the computing device 214 may store the projection data in a storage device 216, such as a hard disk drive, a floppy disk drive, a compact disk-read/write (CD-R/W) drive, a Digital Versatile Disc (DVD) drive, a flash drive, or a solid-state storage device for further evaluation. The storage device 216, or another suitable electronic storage device, may also be employed to store or retain instructions for the operation of one or more functions of the controller 214, including control of the control mechanism 204, in a manner to be described.

In one embodiment, the system 200 may include a user interface or operator console 224, such as a keyboard, mouse and/or touch screen interface, that may be configured to allow user interface and interaction with the system 200 for inputting operational controls to the system 200, as well as for the selection, display and/or modification of images scanning modes, FOV, prior exam data, and/or intervention path. The operator console 224 may also allow on-the-fly access to 2D and 3D scan parameters and selection of an ROI for subsequent imaging, for example, based on operator and/or system commands.

Further, in certain embodiments, the system 200 may be coupled to multiple displays, printers, workstations, a picture archiving and communications system (PACS) 226 and/or similar devices located either locally or remotely, for example, within an institution or hospital, or in an entirely different location via communication links in one or more configurable wired and/or wireless networks such as a hospital network and virtual private networks.

In addition to the C-arm system 102, which can be employed to obtain both pre-operative projection images and/or reconstructed 3D volumetric images 312 and intra-operative 2D images 323 of the patient anatomy, which can subsequently be registered to the pre-op 3D volumetric image(s) 312, the imaging system 200 can additionally include a supplemental imaging system 229, such as an ultrasound imaging system 230 operably connected to the computing device 214. The ultrasound imaging system 230 includes an ultrasound probe 232 connected to the system 230 and capable of obtaining images utilized to acquire a 3D ultrasound image of the patient anatomy. In particular exemplary embodiments, the ultrasound system 230 can produce a 3D ultrasound image utilizing a 3D ultrasound probe, which can be an external or internal (intra-vascular) ultrasound probe, or with a regular 2D ultrasound probe which is navigated, i.e. equipped with navigation sensors providing, in real-time, the location and orientation of the probe 232 in order to enable the 2D images to be processed into a 3D ultrasound image volume of the patient anatomy, or registered to the pre-operative 3D volume 312 of the patient anatomy.

The ultrasound system 230 also includes a system controller 234 that includes a plurality of modules. The system controller 234 is configured to control operation of the ultrasound system 230. For example, the system controller 234 may include an image-processing module 236 that receives the ultrasound signals (e.g., RF signal data or IQ data pairs) and processes the ultrasound signals to generate frames of ultrasound information (e.g., ultrasound images) for displaying to the operator. The image-processing module 236 may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, C-scan, and elastography. The generated ultrasound images may be two-dimensional (2D), three-dimensional (3D) or four-dimensional (4D).

Acquired intra-operative image information, such as fluoroscopic information from the C-arm system 102 or ultrasound information from the ultrasound system 230, may be processed in real-time during an imaging session (or scanning session) as the imaging signals are received. Additionally or alternatively, the intra-operative image information may be stored temporarily in the memory 238 during an interventional procedure and processed in less than real-time in a live or off-line operation. An image memory 240 is included for storing processed frames of intra-operative image information. The image memory 240 may comprise any known data storage medium, for example, a permanent storage medium, removable storage medium, and the like.

In operation, the ultrasound system 230 acquires data, for example, volumetric data sets by various techniques (e.g., 3D scanning, real-time 3D imaging, volume scanning, 2D scanning with transducers having positioning sensors, freehand scanning using a voxel correlation technique, scanning using 2D or matrix array transducers, and the like). The intra-operative images, e.g., ultrasound images, are displayed to the operator or user of the supplemental imaging system 229, e.g., ultrasound system 230, on the display device 218.

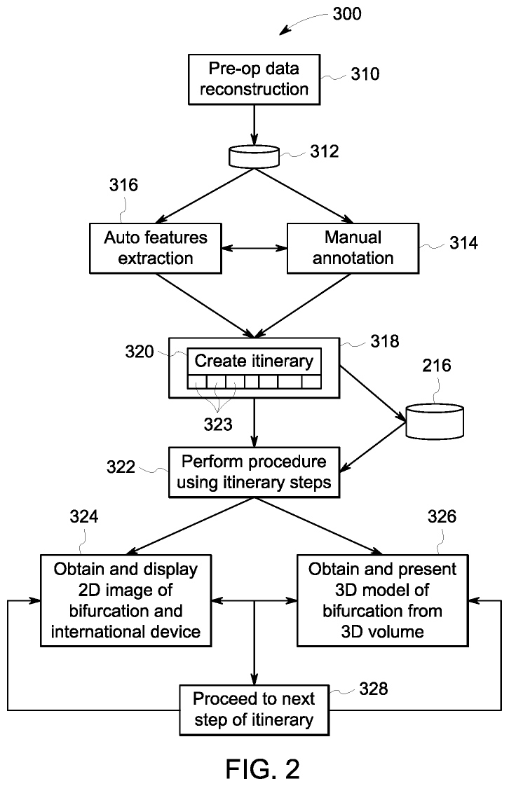

Having provided a description of the general construction of the system 200, the following is a description of a method 300 (see FIG. 2) of operation of the system 200 in relation to the imaged patient anatomy 104. Although an exemplary embodiment of the method 300 is discussed below, it should be understood that one or more acts or steps comprising the method 300 could be omitted or added. It should also be understood that one or more of the acts can be performed simultaneously or at least substantially simultaneously, and the sequence of the acts can vary. Furthermore, it is embodied that at least several of the following steps or acts can be represented as a series of computer-readable program instructions to be stored in the memory 216,238 for execution by the control circuitry/computing device 114,214 for one or more of the radiography imaging system 102 and/or the supplemental e.g., ultrasound, imaging system 230.

In the method 300, in step 310, initially a pre-op image/volume 312, such as pre-op CT image/volume, is obtained of the patient anatomy 104. The CT image/volume 312 is obtained in any suitable imaging manner using the system 102, such as by obtaining a number of projections/projection views of the patient anatomy 104 at various angles, and reconstructing the projection views into the 3D volume 312 representative of the patient anatomy 104, such as by employing the computing device 214 and/or image reconstructor 222 to perform the 3D volume reconstruction from the projection views in a known manner.

In step 314, the 3D volume 312 is presented to the physician on the display 218. Through the user interface 224, the physician can select and review the 3D volume 312 and selected slices thereof in order to provide desired 3D and/or 2D views of the imaged anatomy 104 on the display 218. The system 200 can present the images on the associated display/monitor/screen 218 along with a graphical user interface (GUI) or other displayed user interface. The image may be a software based display that is accessible from multiple locations, such as through a web-based browser, local area network, or the like. In such an embodiment, the image may be accessible remotely to be displayed on a remote device (not shown) in the same manner as the image is presented on the display/monitor/screen 218. Using the user interface/GUI 224, the physician can annotate the selected images, slices, etc., and/or the volume 312 on the display 218 to note the various features and/or structures within the images that are relevant to the interventional procedure to be performed by the physician on the patient anatomy 104, as well as to plan the route 330 (FIG. 3) to be utilized for the interventional device 319 through the patient anatomy 104 to the target tissue(s) or structure(s) 317 in the procedure. The route 330 can be planned according to the structures 313 and/or bifurcations 315 disposed along the route 330 to access the target tissue 317

Concurrently or consecutively with the manual annotation of the 2D and 3D images in step 314, in step 316, the imaging system 200 employs the processor/processing unit/computing device 214 to ascertain the locations of various features present within the 3D volume 312 which can include, but are not limited to, organ(s) and/or vascular structure(s) 313 and any bifurcation(s) 315 contained therein, as well as relevant information 321 concerning them, including but not limited to the diameters and/or tortuosity of the organ and/or vascular structures 313 and bifurcations 315, and/or anomalies or target structures 317 using known identification processes and/or algorithms for CT or other imaging system image generation. For example, traditional image processing techniques, or Artificial Intelligence (AI) based-approaches including machine learning (M L) and deep learning (DL), among others, or a combination of both, which can be employed by the computing device 214 for performing any one or more of the processes or steps of the method 300, such as by utilizing instructions for the operation of the image processing technique and/or AI-based approach stored within the storage device 216 and accessible by the computing device 214, can be used to identify and localize these structures 313, bifurcations 315 and/or anomalies 317 within the 3D volume 312.

After the manual annotation of the images in step 314 and the system analysis of the 3D volume in step 316, the system 100 proceeds to step 318 where the computing device 214 combines the output from step 314, i.e., the manual annotation for the route 330, with the output from step 316, the determination of the location(s) and form of the organ and/or vascular structures 313, the bifurcations 315 and the target tissues 317, as well as the relevant information 321 (FIG. 3) thereon, to form an interventional procedure itinerary 320. In forming the itinerary 320, using traditional image processing techniques, or Artificial Intelligence (AI) based-approaches as described previously, the computing device 214 analyzes the suggested route 330 for the interventional device 319 as determined by the physician in comparison with the information 321 concerning the structures 313 and/or bifurcations 315 forming parts of the route 330 for the interventional device 319. With this information 321, the computing device/AI 214 can confirm, alter and/or suggest alternative paths for the physician-selected route 330 for the interventional device 319 to access the target tissue 317. More specifically, depending upon the information 321 on the features or characteristics (e.g., diameter, tortuosity, etc.) of the structures 313 and bifurcations 315 detected by the computing device/AI 214, the computing device 214 can provide alternative routes 330 to the target tissue 317 that facilitate an easier or simplified route 330 to the target tissue 317. In addition, the computing device/AI 214 can segment the itinerary 320 into individual steps, with each itinerary step 323 corresponding to the traverse of a single structure 313 and/or bifurcation 315 along the route 330.

In addition, the information regarding the structures 313 and bifurcations 315 detected by the computing device/AI 214 enable the computing device/AI 214 to propose alternative forms and/or sizes for the interventional device 319 to be employed in order to accommodate the features, e.g., the diameter and tortuosity, of the structures 313 and/or bifurcations 315 forming the parts or steps 323 of the route 330 for the interventional device 319 to further increase the ease in moving the interventional device 319 along the route 330. In addition, the proposal of alternative interventional devices 319 can enable different and simplified routes 330 to be made available for performing the procedure.

After any accommodations have been made and/or selected for alternative routes and/or devices 319 to be employed in the procedure, the computing device/AI 214 can compile the itinerary 320, which includes step-by-step movements for the interventional device 319 along the route 330 at each bifurcation 315 present along the route. In addition, with the orientation of the structures 313 and bifurcations 315 known within the 3D volume 312 on which the analysis was conducted by the computing device 214, the computing device/AI 214 can also provide information for each itinerary step 323 of the itinerary 320 concerning the position of the C-arm 107 to optimally locate the x-ray source 108 and detector 110 for obtaining the best intra-operative images of the structures 313, bifurcations 315, target tissue(s) 317, and interventional device 319 during the performance of the procedure. The itinerary 320 and associated information, such as the 3D volume 312, the selected interventional device 319 for the procedure, and/or the position of the C-arm 107 for viewing the interventional device 319 at each bifurcation 315, among other information, can be stored in storage device 216 for later use when performing the procedure.

Referring now to FIGS. 2 and 3, during the performance of the procedure, in step 322 the itinerary 320 is accessed by or sent to the imaging system 200 employed for obtaining the intra-operative images 332 during the procedure, which can be the same or different as the imaging system or device 200 used to obtain the pre-operative images used to form the 3D volume 312. Once accessed, in step 324 the information 321 for the current itinerary step 323 of the itinerary 320 is presented on the display 218 in conjunction with the intra-operative image 332 obtained of the bifurcation 315 and the device 319. The information 321 from each itinerary step 323 of the itinerary 320 that can be presented on the display 218 can include, but is not limited to, the location(s) of the target tissue(s) 317 relative to the position of the interventional device 319 shown in the 2D image 332, the predetermined path 325 of the route 330 within the bifurcation 315 shown in the 2D image 332, the information relating to the characteristics, e.g., the diameter, tortuosity and/or path angle, of the bifurcation 315, or portion thereof that forms part of the predetermined path 325, and/or the parameters/position for the optimal visualization angle employed by the imaging system 200/C-arm 107 to obtain the displayed 2D image 332. In addition, the information 321 can include any warnings relating to the current itinerary step 323 of the itinerary 320, such as any notes relating to a required change in the interventional device 319, such as due to a change in the characteristics of the bifurcation 315 from the pre-op image 312 to the intra-op image 332, and/or any pre-op annotations provided by the physician regarding the displayed bifurcation 315.

In addition to presenting the information 321 on the display 218, in step 326, which can be performed concurrently or consecutively with step 324, the imaging system 200 employs the information 321 for the current itinerary step 323 to determine a 3D model 327 of the bifurcation 315 being shown on the display 218. The intra-operative 2D image 332 can be registered to the 3D volume 312, and the bifurcation 315 represented in the 2D image 332 can be recreated in the form of a 3D model 327 presented on the display 218 in conjunction with the 2D image 332. The representation of the 3D model 327 provides the physician with a view of the bifurcation 315 shown in the 2D image 332 in all three dimensions, such that navigation of the interventional device 319 along the predetermined path 325 through the bifurcation 315 is simplified. The presentation of the 3D model 327 on the display 218 can be moved, e.g., rotated in multiple axes, as necessary in order to provide the physician with the view of the model 327 best suited to enable the physician to most readily determine the orientation of the interventional device 319 within the bifurcation 315 and the corresponding direction along which to direct the interventional device 319 to follow the predetermined path 325 though the bifurcation 315 along the planned route 330.

Further, as best shown in FIG. 3, an overlay 340 can be presented on the display 218 in association with the intra-operative 2D image 332. The overlay 340 can contain information relating to the direction of the path 325 for the route 330 through the structure 313 and/or bifurcation 315 represented in the intra-operative 2D image 332, as well as the position of the target tissue(s) 317 relative to the structure 313 and/or bifurcation 315.

When the interventional device 319 has been moved along the bifurcation 315 to a point where the tip 331 of the interventional device 319 is positioned at a specified location, e.g., close to the edge of the 2D image 332, the computing device/AI 214 can proceed to step 328 and move to the next itinerary step 323 of the itinerary 320. In doing so, the computing device/AI 214 accesses the information 321 corresponding to the subsequent itinerary step 323 to determine the location of the bifurcation 315 associated with the next step of the itinerary 320. The computing device/AI 214 then operates the imaging system 200 to obtain a subsequent 2D intra-operative image 332 of the next bifurcation 315 for presentation on the display 218, and optionally for registration with the 3D volume 312, in order to provide the 3D model 327 for presentation in alignment and/or along with the subsequent intra-operative 2D image 332. The computing device/AI 214 can proceed in this manner through each step 323 of the itinerary 320 until all of the pre-determined itinerary steps 323 have been completed and the interventional device 319 has reached the target tissue 317.

With this system and method, for each predetermined step 323 of the itinerary 320 for the particular interventional medical procedure, the physician is provided with each of an intra-operative 2D image 332 obtained, optionally in a continuous manner, at an optimal angle by the imaging system 200 and illustrating the structure 313 and/or bifurcation 315 relating to the particular step 323 of the itinerary 320 and the location of the interventional device 319 within the structure 313 and/or bifurcation 315. Further, in association with each intra-operative 2D image 332, the physician is provided with the information 321 concerning the particular step 323 of the itinerary 320, including the characteristics and structural parameters of the structure 313 and/or bifurcation 315, a manipulatable 3D model 327 illustrating the structure 313 and/or bifurcation 315, and an overlay 340 for the 2D image 323 indicating the portion or path 325 of the route 330 through the structure 313 and/or bifurcation 315 and the location(s) of the target tissue(s) 317 relative to the structure 313 and/or bifurcation 315 being displayed. As such, the physician is provided with detailed information 321 on the characteristics of the structure 313 and/or bifurcation 315 constituting each step of the itinerary 320 as well as information concerning the proper direction for the interventional device 319 along the path 325 and route 330 through the structure 313 and/or bifurcation 315 to perform the interventional procedure.

In an alternative embodiment of the system and method of the present disclosure, the method 300 can be performed automatically by the imaging system 200 and a suitable robotic arm 250, that can be operably connected to the C-arm 107, or can be formed as a free standing structure (not shown). The robotic arm 250 is operably connected to the computing device/AI 214 and includes the interventional device 319 disposed on one end thereof. In the method 300, with the itinerary 320 planned in step 318 by the computing device/AI 214 employing analysis of the 3D volume 312 performed in step 316, the computing device/AI 214 can subsequently control the movement and operation of the C-arm system 102 and the robotic arm 250 to perform each of the itinerary steps 323 and complete the interventional procedure. In this embodiment, the presentation of the 2D image 332 on the display 218 is optional to enable a physician to review the performance of each step 323 of the itinerary 320 of the interventional procedure by the computing device/AI 214.

Finally, it is also to be understood that the system 200 and/or computing device/AI 214 may include the necessary electronics, software, memory, storage, databases, firmware, logic/state machines, microprocessors, communication links, displays or other visual or audio user interfaces, printing devices, and any other input/output interfaces to perform the functions described herein and/or to achieve the results described herein. For example, as previously mentioned, the system may include at least one processor and system memory/data storage structures, which may include random access memory (RAM) and read-only memory (ROM). The at least one processor/computing device/AI 214 of the system 200 may include one or more conventional microprocessors and one or more supplementary co-processors such as math co-processors or the like. The data storage structures discussed herein may include an appropriate combination of magnetic, optical and/or semiconductor memory, and may include, for example, RAM, ROM, flash drive, an optical disc such as a compact disc and/or a hard disk or drive.

Additionally, a software application that adapts the controller/computing device/AI 214, which can be located on the imaging system 200 or remote from the imaging system 200, to perform the methods disclosed herein may be read into a main memory of the at least one processor from a computer-readable medium. The term “computer-readable medium”, as used herein, refers to any medium that provides or participates in providing instructions to the at least one processor of the system 10 (or any other processor of a device described herein) for execution. Such a medium may take many forms, including but not limited to, non-volatile media and volatile media. Non-volatile media include, for example, optical, magnetic, or opto-magnetic disks, such as memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, a RAM, a PROM, an EPROM or EEPROM (electronically erasable programmable read-only memory), a FLASH-EEPROM, any other memory chip or cartridge, or any other medium from which a computer can read.

While in embodiments, the execution of sequences of instructions in the software application causes at least one processor/computing device/AI 214 to perform the methods/processes described herein, hard-wired circuitry may be used in place of, or in combination with, software instructions for implementation of the methods/processes of the present invention. Therefore, embodiments of the present invention are not limited to any specific combination of hardware and/or software.

It is understood that the aforementioned compositions, apparatuses and methods of this disclosure are not limited to the particular embodiments and methodology, as these may vary. It is also understood that the terminology used herein is for the purpose of describing particular exemplary embodiments only, and is not intended to limit the scope of the present disclosure which will be limited only by the appended claims.

What is claimed is:

1. A method for providing guidance for an interventional device during an interventional medical procedure, the method comprising the steps of:
obtaining a pre-operative 3D image volume of a patient anatomy utilizing a first imaging system;
identifying one or more structures, characteristics of the one or more structures, and at least one target tissue in the image volume;
planning an itinerary including a number of steps for insertion of an interventional device through the patient anatomy to the target tissue;
obtaining an intra-operative 2D image of the patient anatomy and interventional device according to one step of the itinerary utilizing a second imaging system; and
registering the intra-operative 2D image to the 3D image volume.

2. The method of claim 1, wherein the first imaging system is selected from the group consisting of a computed tomography (CT) imaging system, a cone beam computed tomography (CBCT) imaging system, and a magnetic resonance imaging (MRI) imaging system.

3. The method of claim 1, further comprising the step of determining a type of interventional device for performing the procedure based on a configuration and the characteristics of the structures along the itinerary.

4. The method of claim 1, wherein the step of planning the itinerary comprises:
determining a route along the one or more structures through the patient anatomy to the target tissue; and
determining the characteristics for each of the one or more structures positioned along the route.

5. The method of claim 4, further comprising the steps of:
forming an overlay of a path forming a portion of the route along the one or more structures present in the 2D image; and
presenting the 2D image on a display in association with the overlay.

6. The method of claim 4, wherein the step of determining a route along the one or more structures in the patient anatomy to the target tissue is performed manually.

7. The method of claim 4, wherein the step of determining the characteristics for each of the one or more structures positioned along the route is performed automatically.

8. The method of claim 7, further comprising the step of altering the route after determining the characteristics for the one or more structures positioned along the route.

9. The method of claim 7, further comprising the step of determining a form of the interventional device to be moved along the route after determining the characteristics for the one or more structures positioned along the route.

10. The method of claim 4, wherein the one or more structures are bifurcations, and wherein the step of determining the route along the one or more structures through the patient anatomy to the target tissue comprises:
determining the locations of the bifurcations along the route; and
forming an individual itinerary step for each bifurcation.

11. The method of claim 10, wherein the step of determining the characteristics for each of the one or more structures positioned along the route comprises determining at least one of a diameter, a tortuosity, an optimal visualization angle, a path angle, and combinations thereof.

12. The method of claim, 11 further comprising the step of presenting the 2D image on a display in association with the characteristics of the bifurcation represented in the 2D image.

13. The method of claim 1, further comprising the steps of:
forming a 3D model of the structure in the 2D image; and
presenting the 2D image on a display in association with the 3D model.

14. The method of claim 1, wherein the step of obtaining an intra-operative 2D image comprises obtaining a first intra-operative 2D image of the patient anatomy and interventional device according to a first step of the itinerary, and wherein the method further comprises the steps of:
moving the interventional device along the patient anatomy represented in the first intra-operative 2D image;
obtaining a second intra-operative 2D image of the patient anatomy and interventional device according to a second step of the itinerary.

15. The method of claim 1, wherein the first imaging system and the second imaging system are the same.

16. An imaging system for providing guidance for movement of an interventional device in an interventional medical procedure, the imaging system comprising:
a first imaging system for obtaining a pre-operative 3D image volume of a patient anatomy;
a second imaging system for obtaining an intra-operative 2D image of the patient anatomy; and
a computing device operably connected to the first imaging system and to the second imaging system, the computing device configured to identify one or more structures, characteristics of the one or more structures, and at least one target tissue in the image volume, to plan an itinerary including a number of steps for insertion of an interventional device through the patient anatomy to the target tissue, and to register the intra-operative 2D image to the 3D image volume.

17. The imaging system of claim 16, wherein the computing device is configured to form a 3D model of the structure in the intra-operative 2D image and present the intra-operative 2D image on a display in association with the 3D model.

18. The imaging system of claim 16, wherein the computing device is configured to plan the itinerary by determining a route along the one or more structures through the patient anatomy to the target tissue and to determining the characteristics for each of the one or more structures positioned along the route.

19. The imaging system of claim 16, wherein the computing device is configured to form an overlay of a path forming a portion of the route along the one or more structures present in the intra-operative 2D image and to presenting the intra-operative 2D image on a display in association with the overlay.

20. The imaging system of claim 16, wherein the one or more structures are bifurcations, and wherein the computing device is configured to determine the route along the one or more structures through the patient anatomy to the target tissue by determining the locations of the bifurcations along the route, and forming an individual itinerary step for each bifurcation.
